(19) United States
(12) Patent Application Publication
(10) Pub. No.: US 2015/0121276 A1
(43) Pub. Date: Apr. 30, 2015

(54) METHOD OF DISPLAYING MULTI MEDICAL IMAGE AND MEDICAL IMAGE EQUIPMENT FOR PERFORMING THE SAME

(71) Applicant: SAMSUNG ELECTRONICS CO., LTD., Suwon-si (KR)

(72) Inventors: Ji-won RYU, Suwon-si (KR); Jae-guyn LIM, Seongnam-si (KR); Woo-young JANG, Seongnam-si (KR); Jae-duck JANG, Suwon-si (KR)

(73) Assignee: SAMSUNG ELECTRONICS CO., LTD., Suwon-si (KR)

(21) Appl. No.: 14/523,144

(22) Filed: Oct. 24, 2014

(30) Foreign Application Priority Data
Oct. 25, 2013 (KR) 10-2013-0128027

(51) Int. Cl. G06F 19/00; G06F 3/0482; G06F 3/0481; A61B 5/00

(52) U.S. Cl. CPC G06F 19/321; A61B 5/7425; G06F 3/0482; G06F 3/04817
USPC 715/771

(57) ABSTRACT

A method of displaying a multi-medical image is provided. The method includes: displaying a multi-medical image user interface including an optical coherence tomography (OCT) image of a target region and at least one other type of medical image of the target region; receiving a first command for storing a first OCT image; storing the first OCT image after receiving the first command; marking a first position corresponding to the stored first OCT image on the at least one other type of medical image displayed on the multi-medical image user interface; and displaying the stored first OCT image on the multi-medical image user interface.

### RELATED APPLICATIONS

This application claims priority from Korean Patent Application No. 10-2013-0128027, filed on Oct. 25, 2013, in the Korean Intellectual Property Office, the disclosure of which is incorporated herein in its entirety by reference.

### BACKGROUND

1. Field

Exemplary embodiments relate to a method of displaying a multi-medical image including an optical coherence tomography (OCT) image and at least one different medical image.

2. Description of the Related Art

Methods and apparatuses for identifying an internal structure of an object, such as the human body or various materials, are widely used in a variety of fields. Examples thereof are various internal transmission image and tomography image capturing equipment, such as an X-ray system, a computerized tomography (CT) scanner, a magnetic resonance image (MRI) apparatus, and an ultrasound system. The equipment may enable a user to identify the cause, position, and development state of various diseases without requiring an incision into the internal structure of the human body or any living body, thereby playing an important role in medical fields. Accordingly, such equipment may be less harmful to living bodies.

An OCT apparatus photographs an internal structure of an object based on an interference phenomenon between reference light and light irradiated to and then reflected from an object. OCT enables the acquiring of a high-resolution image and is harmless to the human body. Due to these aspects, OCT is widely used in medical fields.

OCT is used alone or in combination with an endoscope apparatus, an X-ray apparatus, or an angiography apparatus. These apparatuses have different characteristics, provide different information to users, and have different strengths and weaknesses. For example, OCT provides a precise transmission image of a relatively narrow region and an endoscope apparatus provides an image of an overall appearance of a wider region than the region provided by OCT.

Accordingly, the combining of different medical image apparatuses may overcome their respective weaknesses.

### SUMMARY

Exemplary embodiments include a multi-medical image that is displayed as a combination of an optical coherence tomography image and at least one different medical image to provide an efficient medical image with various pieces of information to a user.

Additional aspects will be set forth in part in the description which follows and, in part, will be apparent from the description, or may be learned by practice of the exemplary embodiments.

According to an exemplary embodiment, a method of displaying a multi-medical image includes displaying a multi-medical image user interface including an optical coherence tomography (OCT) image of a target region and at least one other type of medical image of the target region; receiving a first command for storing a first OCT image; storing the first OCT image after receiving the first command; marking a first position corresponding to the stored first OCT image on the at least one other type of medical image displayed on the multi-medical image user interface; and displaying the first stored OCT image on the multi-medical image user interface.

The receiving may include receiving a second command for storing a second OCT image. The storing may include storing the second OCT image after receiving the second command. The marking may include marking a second position corresponding to the stored second OCT image on the at least one other type of medical image displayed on the multi-medical image user interface.

The first and second positions corresponding to the stored first and second OCT images may be marked on the at least one other type of medical image according to a storage order of the stored first and second OCT images.

The stored first OCT image may be displayed in the form of a thumbnail on the multi-medical image user interface.

The stored first OCT image and at least one other type of medical image having a marking corresponding to the stored first OCT image may be displayed in the form of a thumbnail on the multi-medical image user interface.

The displaying may include displaying the stored first OCT image in an original size in response to selecting the thumbnail corresponding to the stored first OCT image on the multi-medical image user interface.

The at least one other type of medical image may have a wider field of view than the OCT image.

The at least one other type of medical image may be an image selected from an endoscopy image, an X-ray photographing image, and an angiogram image.

The OCT image displayed may be an image registered with other medical images.

According to an exemplary embodiment, a medical image equipment for displaying a multi-medical image includes a medical image acquiring device configured to acquire an optical coherence tomography (OCT) image of a target region and at least one other type of medical image of the target region; a display configured to display the OCT image and the at least one other type of medical image acquired by the medical image acquiring unit; a user interface configured to receive a first command for storing a first OCT image; a storage configured to store the first OCT image displayed at a time of receiving the first command; and a controller configured to control the display to display a multi-medical image user interface comprising the OCT image, the at least one other type of medical image, and the stored first OCT image, wherein the controller is configured to mark a first position corresponding to the stored first OCT image on the at least one other type of medical image displayed on the multi-medical image user interface.

The user interface may be configured to receive a second command for storing a second OCT image, the storage may be configured to store the second OCT image after receiving the second command, and the controller may be configured to mark a second position corresponding to the stored second OCT image on the at least one other type of medical image displayed on the multi-medical image user interface.

The controller may mark the first and second positions corresponding to the stored first and second OCT images according to a storage order of the stored first and second OCT images on the at least one other type of medical image.

The controller may control the display to display the stored OCT first image in the form of a thumbnail on the multi-medical image user interface.

The controller may control the display to display the stored first OCT image and at least one other type of medical image having a marking corresponding to the stored first OCT image in the form of a thumbnail on the multi-medical image user interface.

The controller may control the display to display the stored first OCT image in its original size on the multi-medical image user interface in response to a user selection of the thumbnail corresponding to the stored first OCT image on the multi-medical image user interface.

The at least one other type of medical image may have a wider field of view than the OCT image.

The at least one other type of medical image may be an image selected from an endoscopy image, an X-ray photographing image, and an angiogram image.

The OCT image displayed may be an image registered with an endoscopy image.

### BRIEF DESCRIPTION OF THE DRAWINGS

These and/or other aspects will become apparent and more readily appreciated from the following description of the exemplary embodiments, taken in conjunction with the accompanying drawings in which:

FIG. 1 is a view illustrating the configuration of a medical image equipment for displaying a multi-medical image (e.g., a multi-medical image user interface), according to an exemplary embodiment;

FIG. 2 is a view of a multi-medical image according to an exemplary embodiment;

FIGS. 3A to 3D are views for explaining how an optical coherence tomography (OCT) image of a multi-medical image is stored according to an exemplary embodiment;

FIGS. 4 to 6 are views showing multi-medical images according to exemplary embodiments; and

FIG. 7 is a flowchart illustrating a method of displaying a multi-medical image according to an exemplary embodiment.

### DETAILED DESCRIPTION OF EXEMPLARY EMBODIMENTS

Reference will now be made in detail to exemplary embodiments, which are illustrated in the accompanying drawings, wherein like reference numerals refer to like elements throughout. In this regard, the exemplary embodiments may have different forms and should not be construed as being limited to the descriptions set forth herein. Accordingly, the exemplary embodiments are merely described below, by referring to the figures, to explain aspects of the exemplary embodiments.

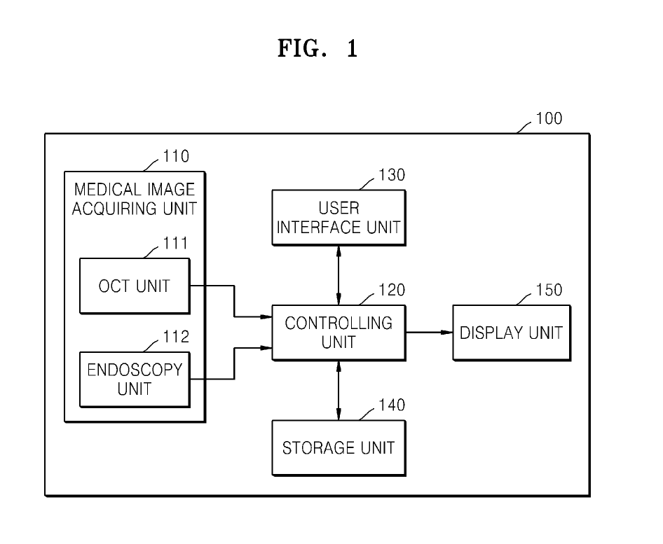

FIG. 1 is a view illustrating the configuration of a medical image equipment 100 for displaying a multi-medical image, according to an exemplary embodiment. Referring to FIG. 1, the medical image equipment 100 according to an exemplary embodiment includes a medical image acquiring unit 110 (e.g., a medical image acquirer), a controlling unit 120 (e.g., a controller), a user interface unit 130 (e.g., a user interface), a storage unit 140 (e.g., a storage), and a display unit 150 (e.g., a display). The medical image acquiring unit 110 may include an optical coherence tomography (OCT) unit 111 (e.g., optical coherence tomographer) and an endoscopy unit 112 (e.g., an endoscopy device). Although the medical image acquiring unit 110 according to an exemplary embodiment includes the endoscopy unit 112, the medical image acquiring unit 110 may include, instead of or in addition to the endoscopy unit 112, an X-ray photographing unit (e.g., an X-ray device or machine) or an angiography unit (e.g., an angiography device or machine).

The optical coherence tomography (OCT) unit 111 and the endoscopy unit 112 of the medical image acquiring unit 110 photographs a common region. The OCT unit 111 may acquire an OCT image in real time by using interference of light reflected from a target region, and then transmit the obtained real-time OCT image to the controlling unit 120. The endoscopy unit 112 photographs the same target region to acquire an endoscopy image thereof, which is then transmitted to the controlling unit 120.

In this regard, since the field of view of the endoscopy image is wider than that of the OCT image, the endoscopy image may contain information of a wider tissue region. An X-ray photographing unit or an angiography unit may be used instead of the endoscopy unit 112. Since an X-ray image and an angiogram image also have a wider field of view than that of the OCT image, the X-ray image and the angiogram image may contain information of a wider tissue region. As described above, the medical image acquiring unit 110 may acquire an OCT image having precise information about the internal structure of a relatively narrow region and at least one different medical image information that has a lower degree of precision and is about a wider tissue region than the OCT image.

The controlling unit 120 may allow the display unit 150 to display the real-time OCT image and the endoscopy image, which are received from the medical image acquiring unit 110, as a multi-medical image on a single screen. That is a multi-medical image displayed on a single screen are divided into several regions and in the respective regions, a real-time OCT image and an endoscopy image may be displayed. While not required in all aspects, the controlling unit 120 can include a processor or microprocessor executing a computer program stored in a computer-readable medium, such as the storage unit 140.

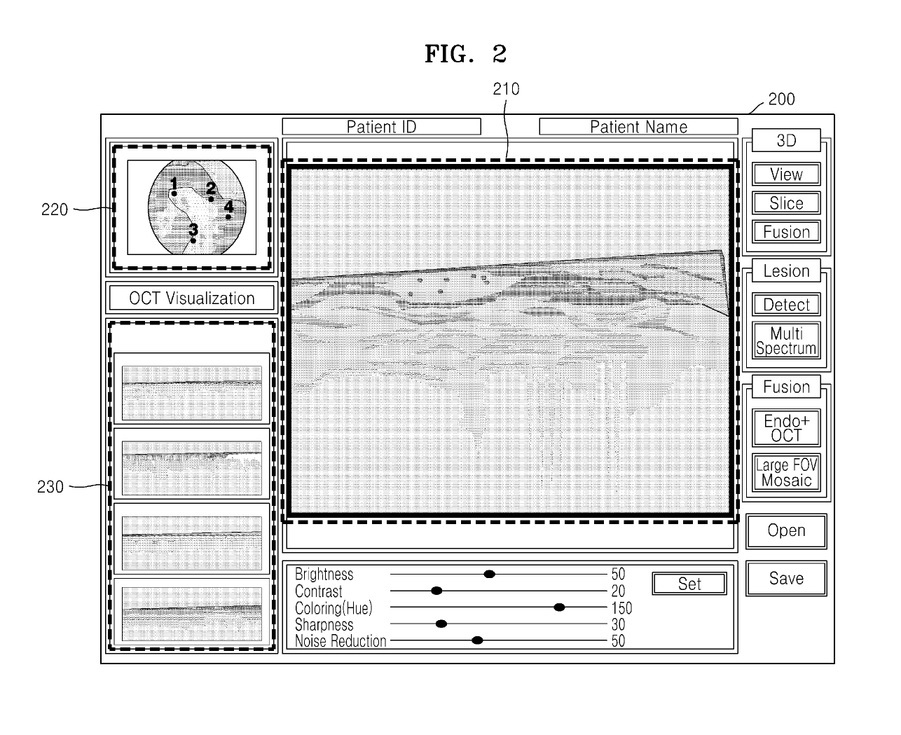

A detailed example of the multi-medical image including the real-time OCT image and the endoscopy image will be described in detail with reference to FIGS. 2 to 7.

When a user inputs a command for storing the OCT image, the controlling unit 120 controls storing the OCT image, displaying the stored OCT image, and marking a position corresponding to the OCT image on the endoscopy image. The position that is marked may correspond to a location of the OCT image with respect to or within the endoscopy image or other type of medical image. In detail, when a user inputs a command for storing the OCT image by using the user interface unit 130, from among OCT images being displayed in real time on the display unit 150, an OCT image displayed at a time when the command is input, is stored in the storage unit 140. Also, the controlling unit 120 marks a position corresponding to the OCT image stored in the storage unit 140 on the endoscopy image displayed on the display unit 150. The position that is marked may correspond to a location of the OCT image with respect to or within the endoscopy image or other type of medical image.

The storage unit 140 may store a plurality of OCT images, and when a plurality of OCT images are stored, the controlling unit 120 may mark positions corresponding to the OCT images on the endoscopy image respectively. For example, positions corresponding to OCT images may be marked with different colors, or may be labeled with numbers determined according to the storage order. According to another exemplary embodiment, positions corresponding to OCT images may be distinguishably marked by using other methods. The storage unit may include memory that may be external or internal, and may be a volatile memory (such as RAM) or a non-volatile memory (such as ROM, flash memory, or a hard disk drive).

The controlling unit 120 may display the OCT image stored in the storage unit 140 on the multi-medical image displayed on the display unit 150. That is, the real-time OCT image acquired by the medical image acquiring unit 110 is displayed on a single screen together with the stored OCT image that has been stored with an endoscopy image to provide various pieces of information to a user. In this regard, the controlling unit 120 may display the stored OCT image in the form of a thumbnail on the multi-medical image. By doing so, the stored OCT image occupies a relatively small space, and accordingly, a plurality of stored OCT images may be effectively displayed on a single screen. Also, together with the stored OCT image, an endoscopy image on which a position corresponding to the stored OCT image is marked may also be displayed in the form of a thumbnail. When a user selects a stored OCT image displayed in the form of a thumbnail, the selected OCT image may be displayed in its original size on a screen.

A detailed example of a multi-medical image displayed on a screen and detailed operations performed when an OCT image is stored on a multi-medical image and the stored OCT image is displayed will be described in detail with reference to FIGS. 2 to 7.

FIG. 2 shows a multi-medical image displayed on a screen of a medical image equipment according to an exemplary embodiment. Referring to FIG. 2, the multi-medical image 200 may have a first region 210 displaying a real-time OCT image, a second region 220 displaying an endoscopy image, and a third region 230 displaying stored OCT images in the form of a thumbnail.

In an exemplary embodiment, an endoscopy image is displayed on the second region 220. In other exemplary embodiments, however, an X-ray photographing image or an angiogram image may be displayed. That is, on the second region 220, a different medical image of a target region, of which a real-time OCT image is displayed on the first region 210, may be displayed. In this regard, since the medical image displayed on the second region 220 has a wider field of view than the OCT image, a position corresponding to the OCT image may be marked on the medical image displayed on the second region 220. The positions corresponding to the OCT image may be marked with graphical objects or identifiers that are selectable to display the corresponding OCT image.

While watching a real-time OCT image displayed on the first region 210, the user may input a command for storing an OCT image in a medical image equipment to store the OCT image. When the user inputs a storage command for storing the OCT image, the OCT image displayed at the time when the storage command is input, is stored in the medical image equipment and a position corresponding to the stored OCT image is marked on the endoscopy image displayed on the second region 220. A stored OCT image may be displayed in the form of a thumbnail on the third region 230. When a plurality of OCT images are stored, positions corresponding to OCT images may be distinguishably marked on the endoscopy image of the second region 220, and on the third region 230, OCT images may be displayed in the form of a thumbnail.

The position and size of the respective regions marked on the multi-medical image 200 illustrated in FIG. 2 are an example only, and may vary according to purpose or user's convenience.

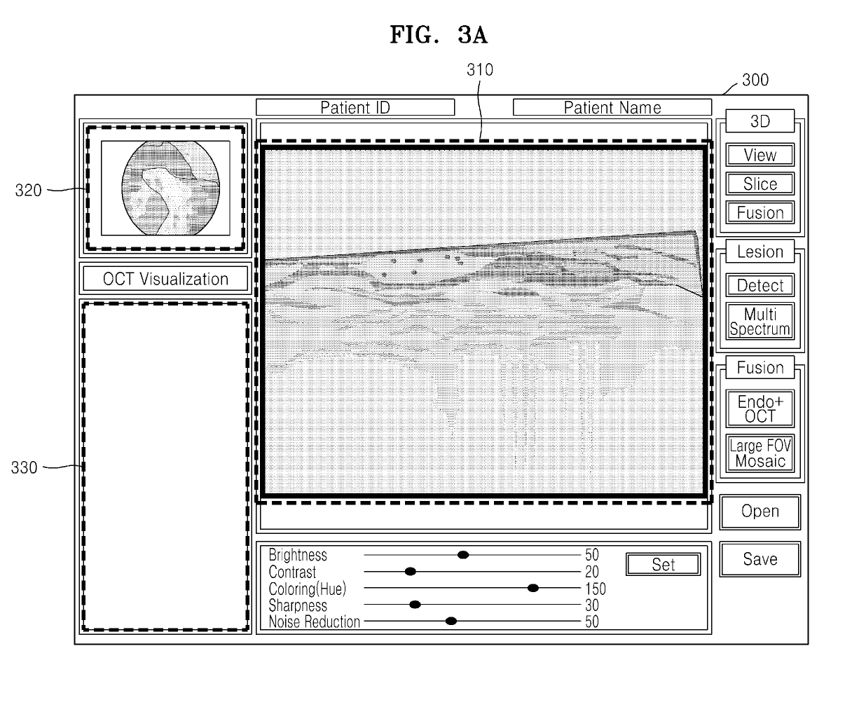

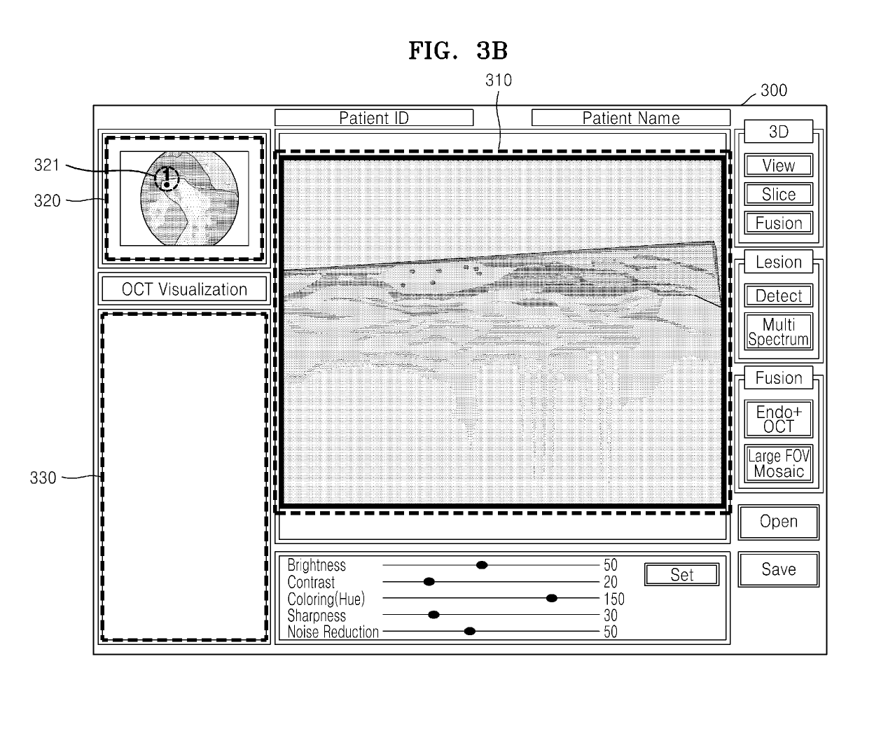

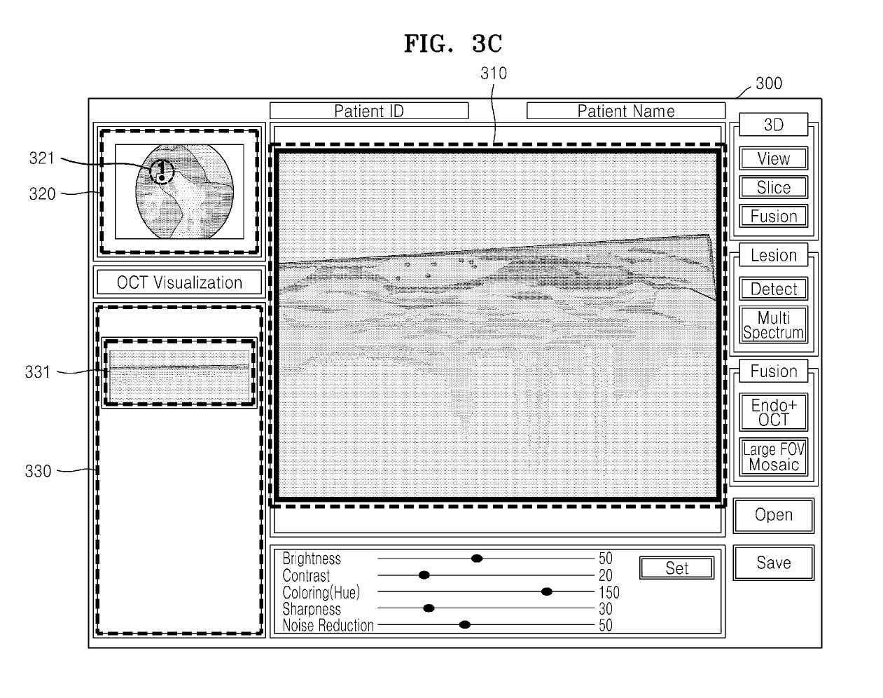

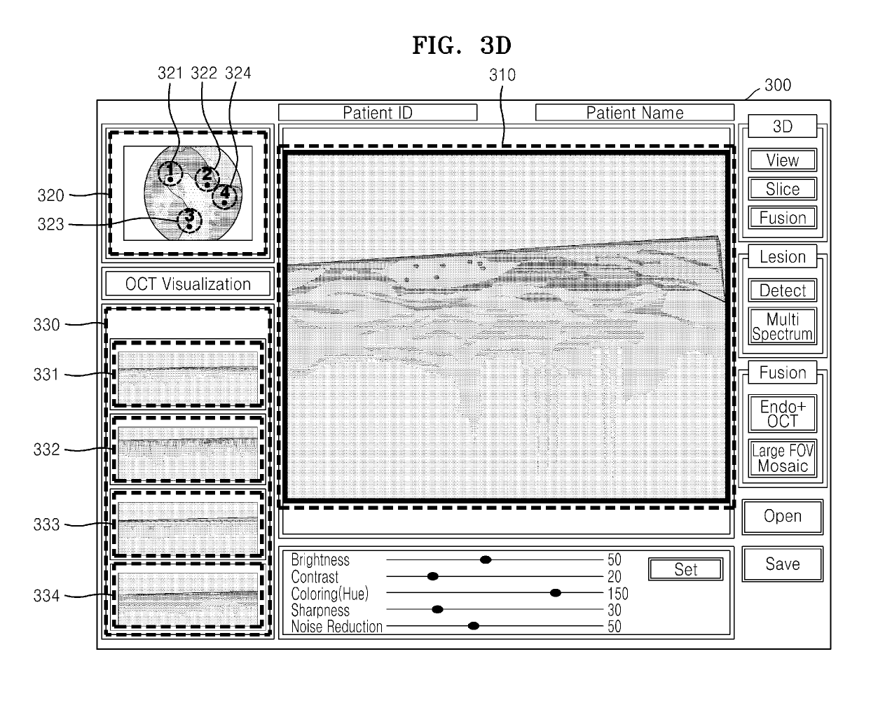

FIGS. 3A to 3D are views for explaining how an OCT image of a multi-medical image 300 is stored according to an exemplary embodiment.

Referring to FIG. 3A, the multi-medical image 300 has a first region 310 displaying a real-time OCT image of a target region, a second region 320 displaying an endoscopy image of the target region, and a third region 330 displaying nothing since OCT images have not yet been stored.

Referring to FIG. 3B, when an OCT image is stored, a position corresponding to the OCT image is marked on the endoscopy image displayed on the second region 320. When a user watches a real-time OCT image displayed on the first region 310 and the user determines to store the real-time OCT image and inputs a storage command, the OCT image displayed on the first region 310 at the time of inputting of the command is stored in the medical image equipment. As illustrated in FIG. 3B, a position corresponding to the OCT image is marked with a marking 321 on the endoscopy image displayed on the second region 320.

Referring to FIG. 3C, a stored OCT image is displayed in the form of a thumbnail on the third region 330. The stored OCT image illustrated in FIG. 3C is displayed as a thumbnail 331 on the third region 330. Accordingly, the user may obtain an overall view of the OCT image by viewing the thumbnail 331 displayed on the third region 330. Since a stored OCT image is displayed in the form of a small thumbnail, an OCT image may be displayed with other medical images while occupying a relatively small space.

The multi-medical image 300 of FIG. 3D includes a plurality of OCT images that have been stored. Referring to FIG. 3D, an endoscopy image displayed on the second region 320 has a plurality of markings 321 to 324. Also, the third region 330 displays a plurality of thumbnails 331 to 334. When a plurality of OCT images are stored, positions corresponding to the OCT images are all marked on the endoscopy image displayed on the second region 320, and stored OCT images are displayed in the form of a thumbnail on the third region 330.

In this regard, a plurality of markings 321 to 324 on the endoscopy image of the second region 320 are distinguishable from each other so that a user identifies what marking corresponds to what stored OCT image. For example, the markings 321 to 324 of the endoscopy image illustrated in FIG. 3 are labeled with the number from 1 to 4. These numbers may indicate a storage order of OCT images. That is, the thumbnails 331 to 334 displayed on the third region 330 are located in an upper position, and the marking 321 marked with the number “1” corresponds to the thumbnail 331 located in the most upper position, and the marking 322 marked with the number “2” corresponds to the thumbnail 332 located under the thumbnail 331.

In an exemplary embodiment, markings are distinguishable by using color instead of numbering. For example, the color of a marking marked on the endoscopy image displayed on the second region 320 may be identical to the color of a corresponding thumbnail so that a user identifies what marking corresponds to what stored OCT image.

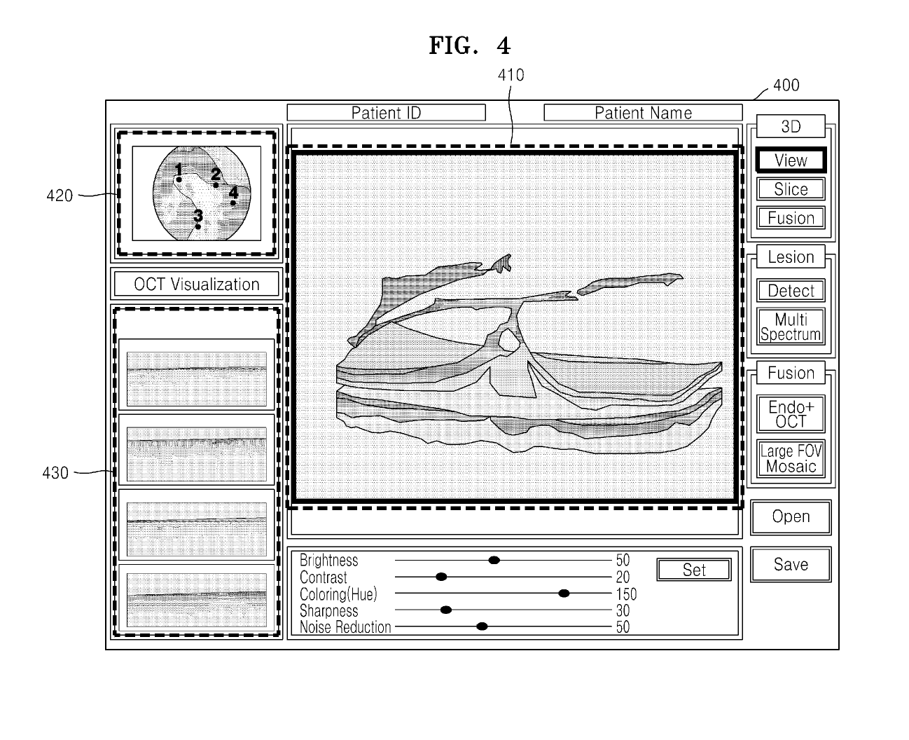

FIGS. 4 to 6 are views illustrating multi-medical images according to various exemplary embodiments.

FIG. 4 shows that a real-time OCT image of a multi-medical image 400 displayed on a first region 410 is three-dimensional (3D). From the 3D real-time OCT image, the user may obtain specific and dimensional information about an internal structure. Meanwhile, an endoscopy image is displayed on a second region 420, and a plurality of thumbnails of stored OCT images are displayed on a third region 430. The endoscopy image displayed on the second region 420 has markings 1 to 4 indicating positions corresponding to the thumbnails displayed on the third region 430.

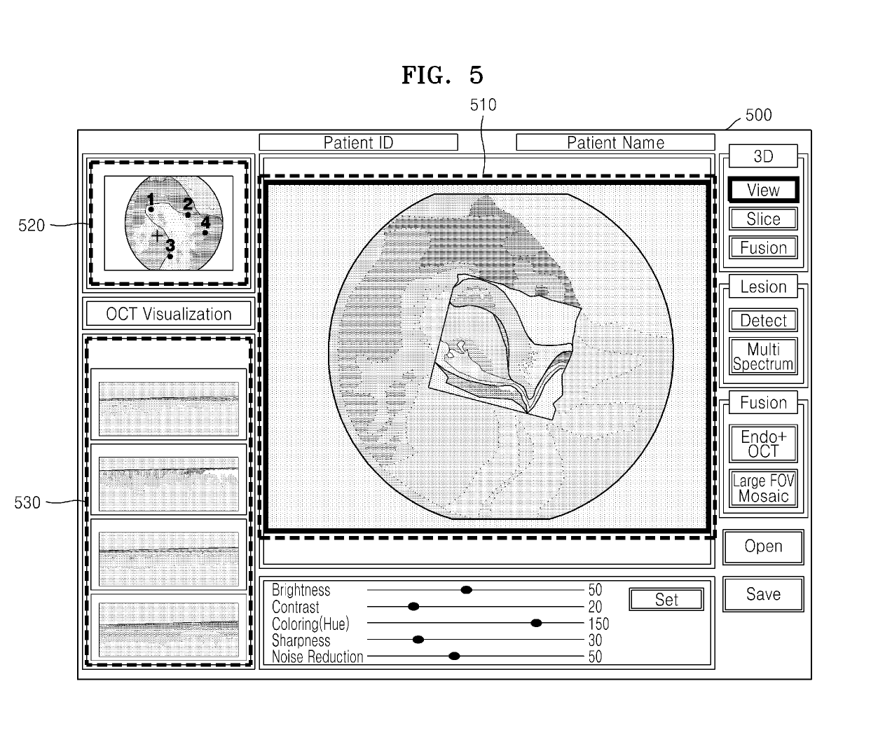

FIG. 5 shows that a real-time OCT image of a multi-medical image 500 displayed on a first region 510 is an image registered with an endoscopy image. From the registered image, the user may simultaneously identify a position inside the tissue corresponding to an OCT image and detailed OCT images in real-time. Meanwhile, an endoscopy image is displayed on a second region 520, and a plurality of thumbnails of stored OCT images are displayed on a third region 530. The endoscopy image displayed on the second region 520 has markings 1 to 4 indicating positions corresponding to the thumbnails displayed on the third region 530.

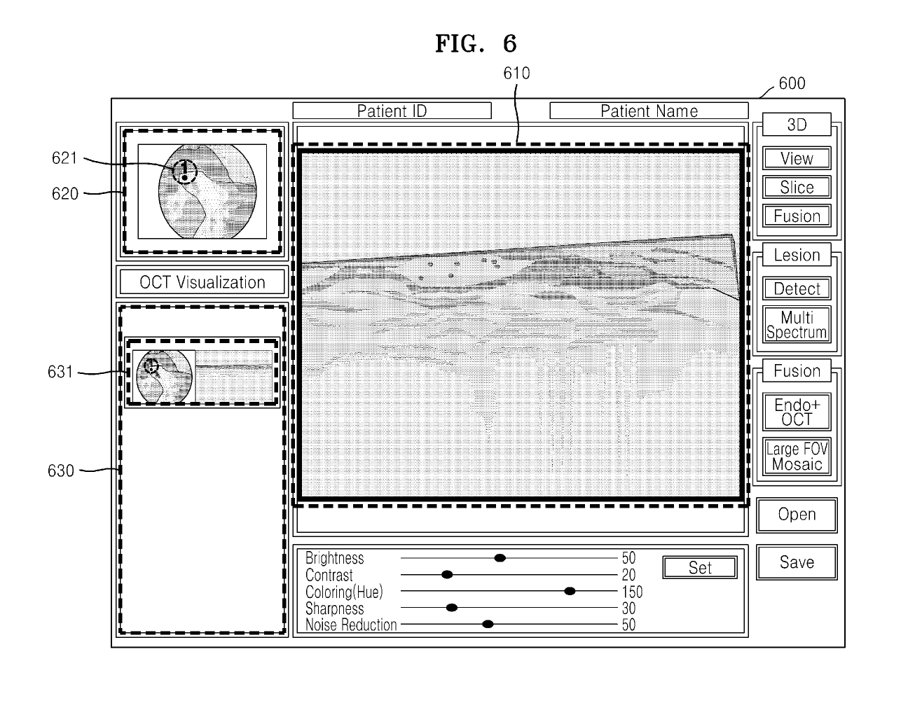

FIG. 6 shows that a third region 630 of a multi-medical image 600 displays a thumbnail including a stored OCT image and an endoscopy image having a marking corresponding to the OCT image. Referring to FIG. 6, a thumbnail 631 displayed on the third region 630 includes a stored OCT image and an endoscopy image having a marking corresponding to the OCT image. For example, thumbnail 631 includes the marking 1 to indicate that the thumbnail 631 corresponds to marking 621 in second region 620. Accordingly, the user may identify a stored OCT image and a position corresponding thereto only with the thumbnail 631 displayed on the third region 630. Meanwhile, a real-time OCT image is displayed on a first region 610.

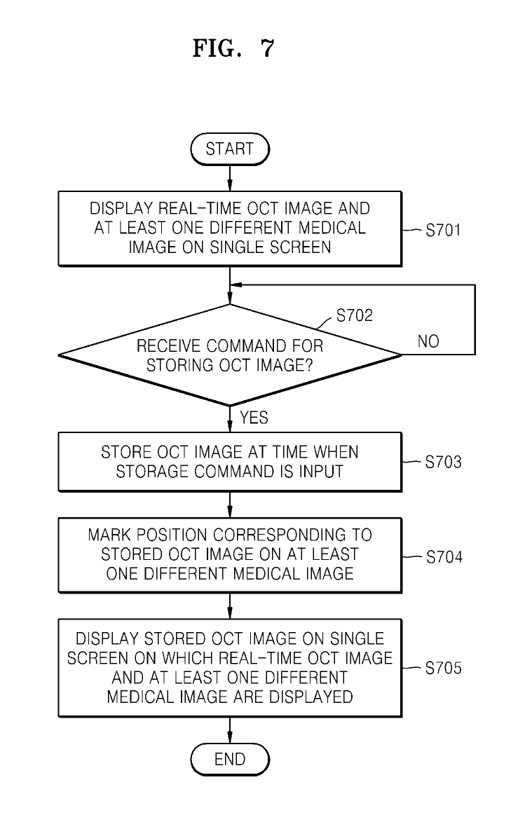

FIG. 7 is a flowchart illustrating a method of displaying a multi-medical image according to an exemplary embodiment. Hereinafter, a method of displaying a multi-medical image according to an exemplary embodiment will be described in detail with reference to FIG. 7.

Referring to FIG. 7, in operation of S701, a real-time OCT image and at least one different type of medical image are displayed on a single screen. That is, a multi-medical image including both a real-time OCT image and at least one different type of medical image is displayed on a display unit of the medical image equipment. In this regard, the different type of medical image may be an endoscopy image, an X-ray photographing image, or an angiogram image, and a target region of the image may be identical to that of a real-time OCT image. Also, the different type of medical image may have a wider field of view than the OCT image.

In operation 5702, whether an OCT storage command is input by the user is determined, and once determined the receiving, operation 5703 is performed to store an OCT image displayed at the time when the command is input is stored in the medical image equipment. Then, in operation 5704, a position corresponding to a stored OCT image is marked on at least one different medical image. In this regard, the marking may be indicated with a variety of color or by numbering, and when a plurality of OCT images are stored, positions corresponding to a plurality of OCT images may be distinguishably marked.

Finally, in operation 5705, the stored OCT image is displayed on the single screen on which the real-time OCT image and the at least one different type of medical image are displayed. In this regard, the stored OCT image may be displayed in the form of a thumbnail, and may be displayed image in the form of a thumbnail together with at least one different medical image having a marking corresponding to the stored OCT. Also, when a plurality of OCT images are stored, thumbnails corresponding to a plurality of OCT images may be all displayed.

According to an exemplary embodiment, a real-time OCT image and at least one different type of medical image or a medical image from a different medical imaging device of a common target region are simultaneously displayed on a single screen, and when an OCT image is stored, a position corresponding to a stored OCT image is marked on the different medical image, and the marked medical image and the stored OCT image are displayed on the same screen, and by doing so, a user may be provided with an anatomical image together with additional positional information and tissue characteristics, leading to a help for lesion diagnosis.

While not restricted thereto, an exemplary embodiment can be embodied as computer-readable code on a computer-readable recording medium. The computer-readable recording medium is any data storage device that can store data that can be thereafter read by a computer system. Examples of the computer-readable recording medium include read-only memory (ROM), random-access memory (RAM), CD-ROMs, magnetic tapes, floppy disks, and optical data storage devices. The computer-readable recording medium can also be distributed over network-coupled computer systems so that the computer-readable code is stored and executed in a distributed fashion. Also, an exemplary embodiment may be written as a computer program transmitted over a computer-readable transmission medium, such as a carrier wave, and received and implemented in general-use or special-purpose digital computers that execute the programs.

It should be understood that the exemplary embodiments described therein should be considered in a descriptive sense only and not for purposes of limitation. Descriptions of features or aspects within each exemplary embodiment should typically be considered as available for other similar features or aspects in other embodiments.

While one or more exemplary embodiments have been described with reference to the figures, it will be understood by those of ordinary skill in the art that various changes in form and details may be made therein without departing from the spirit and scope of the inventive concept as defined by the following claims.

What is claimed is:

1. A method of displaying a multi-medical image, the method comprising
displaying a multi-medical image user interface comprising an optical coherence tomography (OCT) image of a target region and at least one other type of medical image of the target region;
receiving a first command for storing a first OCT image;
storing the first OCT image after receiving the first command;
marking a first position corresponding to the stored first OCT image on the at least one other type of medical image displayed on the multi-medical image user interface; and
displaying the first stored OCT image on the multi-medical image user interface.

2. The method of claim 1, wherein the receiving comprises receiving a second command for storing a second OCT image,
wherein the storing comprises storing the second OCT image after receiving the second command, and
wherein the marking comprises marking a second position corresponding to the stored second OCT image on the at least one other type of medical image displayed on the multi-medical image user interface.

3. The method of claim 2, wherein
the first and second positions corresponding to the stored first and second OCT images are marked on the at least one other type of medical image according to a storage order of the stored first and second OCT images.

4. The method of claim 1, wherein the stored first OCT image is displayed in the form of a thumbnail on the multi-medical image user interface.

5. The method of claim 4, wherein the stored first OCT image and at least one other type of medical image having a marking corresponding to the stored first OCT image are displayed in the form of a thumbnail on the multi-medical image user interface.

6. The method of claim 4, further comprising
displaying the stored first OCT image in an original size in response to selecting the thumbnail corresponding to the stored first OCT image on the multi-medical image user interface.

7. The method of claim 1, wherein the at least one other type of medical image has a wider field of view than the OCT image.

8. The method of claim 1, wherein the at least one other type of medical image is an image selected from an endoscopy image, an X-ray photographing image, and an angiogram image.

9. The method of claim 1, wherein the OCT image displayed is an image registered with other medical images.

10. A non-transitory computer-readable recording medium having embodied thereon a program for executing the method of claim 1.

11. A medical image equipment for displaying a multi-medical image, the medical image equipment comprising:
a medical image acquirer configured to acquire an optical coherence tomography (OCT) image of a target region and at least one other type of medical image of the target region;
a display configured to display the OCT image and the at least one other type of medical image acquired by the medical image acquiring unit;
a user interface configured to receive a first command for storing a first OCT image;
a storage configured to store the first OCT image displayed at a time of receiving the first command; and
a controller configured to control the display to display a multi-medical image user interface comprising the OCT image, the at least one other type of medical image, and the stored first OCT image,
wherein the controller is configured to mark a first position corresponding to the stored first OCT image on the at least one other type of medical image displayed on the multi-medical image user interface.

12. The medical image equipment of claim 11, wherein the user interface is configured to receive a second command for storing a second OCT image,
wherein the storage is further configured to store the second OCT image after receiving the second command, and
wherein the controller is further configured to mark a second position corresponding to the stored second OCT image on the at least one other type of medical image displayed on the multi-medical image user interface.

13. The medical image equipment of claim 12, wherein
the controller is further configured to mark the first and second positions corresponding to the stored first and second OCT images according to a storage order of the stored first and second OCT images on the at least one other type of medical image.

14. The medical image equipment of claim 11, wherein
the controller is further configured to control the display to display the stored OCT first image in the form of a thumbnail on the multi-medical image user interface.

15. The medical image equipment of claim 14, wherein
the controller is further configured to control the display to display the stored first OCT image and at least one other type of medical image having a marking corresponding to the stored first OCT image in the form of a thumbnail on the multi-medical image user interface.

16. The medical image equipment of claim of claim 14, wherein the controller is further configured to control the display to display the stored first OCT image in its original size on the multi-medical image user interface in response to a user selection of the thumbnail corresponding to the stored first OCT image on the multi-medical image user interface.

17. The medical image equipment of claim of claim 11, wherein the at least one other type of medical image has a wider field of view than the OCT image.

18. The medical image equipment of claim of claim 11, wherein the at least one other type of medical image is an image selected from an endoscopy image, an X-ray photographing image, and an angiogram image.

19. The medical image equipment of claim of claim 11, wherein the OCT image displayed is an image registered with an endoscopy image.

20. A method of displaying a multi-medical image user interface, the method comprising:
displaying an optical coherence tomography (OCT) image of a target region in a first region of the multi-medical image user interface;
displaying at least one other type of medical image of the target region in a second region of the multi-medical image user interface;
receiving a first command to store a first OCT image displayed in the first region;
storing the first OCT image after receiving the first command;
displaying a mark at a first position corresponding to the stored first OCT image on the at least one other type of medical image displayed in the second region; and
displaying a thumbnail of the stored first OCT image in a third region of the multi-medical image user interface.

21. The method of claim 20, wherein the first position is a location in which the first OCT image is captured with respect to the at least one other type of medical image.
